(19) United States
(12) Patent Application Publication
(10) Pub. No.: US 2018/0000977 A1
(43) Pub. Date: Jan. 4, 2018

(54) METHOD AND APPARATUS FOR EXTENDING THE RANGE AND EFFECTIVENESS OF EVAPORATIVE SCENTS

(71) Applicant: Paul D Mitchell, Spencer, MA (US)

(72) Inventor: Paul D Mitchell, Spencer, MA (US)

(21) Appl. No.: 15/200,107

(22) Filed: Jul. 1, 2016

(51) Int. Cl. A61L 9/03; B05B 7/16

(52) U.S. Cl. CPC A61L 9/032; B05B 7/1613; A61L 2209/135

(57) ABSTRACT

A method and apparatus for extending the range and effectiveness of scents is disclosed. The apparatus includes a container which contains a scented substance and a controllable heat source which is used to heat the scented substance to a vaporized state. A controllable air supply is then used to blow the vaporized scent into the environment. The apparatus is controlled by an electronic control unit.

The present invention generally relates to the sense of smell and more particularly, is directed to a method and apparatus for extending the range and effectiveness of evaporative scents.

### BACKGROUND

Scientists and scholars have written that olfaction, or the sense of smell, is the oldest sense. Everything that can be smelled is the result of an evaporative process that produces chemical molecules that float through the air. In humans and air-breathing animals, these molecules enter the nasal cavity and are detected by olfactory receptors. Each receptor is encoded by a specific gene to recognize a different odor. It is believed that humans can distinguish more than 10,000 different odors.

Smell is the only sense that goes directly to the brain. It connects to regions in the brain that affect the nervous system. Smells can relax or stimulate and is the basis for aromatherapy.

Related to the sense of smell are pheromones. Pheromones are chemicals that are secreted through sweat and other body fluids, such as urine. These chemicals are believed to influence and trigger sexual interest and excitement in members of the opposite sex in the same species. Thus, pheromones are said to be capable of acting outside the body of the secreting individual.

It is well known in the art to use natural and artificially created smells and pheromones for various purposes. For example, perfumes, air fresheners, scented candles and the like are commercially successful products that can bring a more pleasing ambiance to a room. It is also well known among hunters to use natural scents and pheromones to attract prey.

Scents and pheromones play an important role in the social behavior and reproductive cycles of many wild animals. For example, territorial animals often use their urine and feces to mark the territorial boundaries that they will defend as their own. Thus, the scent of their urine and feces wards off other animals who might otherwise intrude into the protected territory.

Many animals also use scents and pheromones to communicate with members of the opposite sex. One reason for this is that animals tend to have a much keener sense of smell than do humans and other living creatures. Animals send communications by secreting pheromones which are then received by other animals in the form of smells.

This communication technique is particularly effective in deer species during rut, or mating season. While early researchers theorized that the beginning of rut is triggered by lunar cycles, the currently accepted view is that the onset of rut is determined by the photoperiod.

The photoperiod is the time interval within a twenty-four hour period during which an animal is exposed to light. In many animals, such as deer, the length of the photoperiod regulates the production of hormones that are directly related to the breeding season. As the number of daylight hours decline as fall progresses to winter, the photoperiod correspondingly diminishes in length and thus triggers an increase in certain hormone levels in deer that lead to the mating season.

These hormones are secreted by female deer (doe) as pheromones in urine and other body fluids that evaporate into the air as scents. These scents are picked up by male deer (bucks) and attract them to does for mating. This system of communication operates in a similar manner in other animal species.

It is well known in the art to use pheromones and other wild game scented attractants to attract prey, such as deer and the like, to a particular location. Male prey often is the most responsive to pheromones and other scents related to mating. Thus, the mating season is the most effective time for hunting many species of wild game, especially male prey.

Wick systems, drip systems and aerosols are among the most common ways of dispensing evaporative scents to set the ambience of a room or in the case of hunters, dispense pheromones to attract prey. The effectiveness of these systems is in large measure dependent on the temperature, humidity and air movement in the climate in which they are used.

Prior art approaches and methods of dispensing evaporative scents into the air are not fully effective or operational over a wide range of climate conditions. Accordingly, there is a need in the art for an improved method and apparatus for extending the range and effectiveness of evaporative scents.

### BRIEF DESCRIPTION OF THE DRAWINGS

The novel features of the present invention are set out with particularity in the appended claims, but the invention will be understood more fully and clearly from the following detailed description of the invention as set forth in the accompanying drawings in which:

FIG. 1 is a mechanical block diagram of one embodiment of an electronic vaporizer system in accordance with the present invention;

FIG. 2 is a mechanical block diagram of one embodiment of a manually operated vaporizer system in accordance with the present invention;

FIGS. 3-6 are mechanical block diagrams of the scented liquid tank and vaporizing chamber of the vaporizer systems shown in FIGS. 1 and 2 in accordance with the present invention;

FIG. 7 is a mechanical block diagram of an alternative embodiment of the air pump shown in FIGS. 1 and 2 in accordance with the present invention;

FIG. 8 is a block diagram of one embodiment of a control system used to control the operation of the vaporizer shown in FIG. 1 in accordance with the present invention.

FIGS. 9-10 are block diagrams of one embodiment of a control system used to control the operation of the vaporizer shown in FIG. 2 in accordance with the present invention;

FIG. 11 is a block diagram of a further embodiment of a control system used to control the operation of the vaporizer shown in FIG. 1 in accordance with the present invention;

FIG. 12 is a block diagram of a further embodiment of a control system used to control the operation of the vaporizer shown in FIG. 1 in accordance with the present invention;

FIG. 13 is flow chart illustrating the operation of the control unit illustrated in FIG. 12;

FIG. 14 is a further embodiment of the control unit used to control the operation of the electronic vaporizer shown in FIG. 1; and

FIG. 15 is a still further embodiment of the control unit used to control the operation of the electronic vaporizer shown in FIG. 1.

### BRIEF DESCRIPTION OF THE PREFERRED EMBODIMENT

A preferred embodiment of the present invention will be described with reference to the drawing figures.

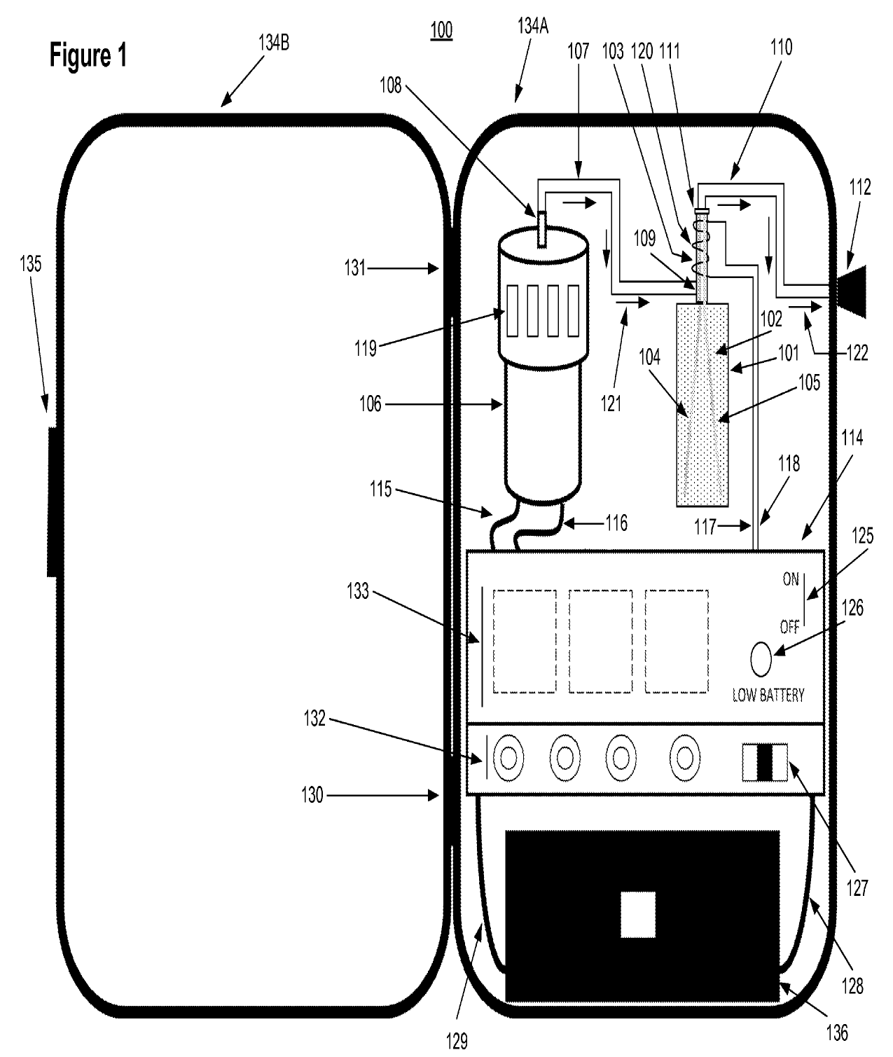

FIG. 1 illustrates one embodiment of an electronic vaporizer 100 for dispensing evaporative scents into the air in accordance with the present invention. The vaporizer can operate in a plurality of modes depending on the requirements at the time. These modes include a continuous mode, a cycle mode and a manual mode. Other modes will be appreciated by those of ordinary skill in the art without departing from the true scope of the present invention.

As shown in FIG. 1, vaporizer 100 includes a chamber or tank 101 which contains, for example, a liquid wild game scent or attractant as generally indicated by reference number 102, to be vaporized and dispensed into the outside air.

Liquid 102 is drawn into vaporizing chamber 103 by wicks 104 and 105. The wicks are made of a porous material through which liquid 102 is drawn by capillary action into chamber 103. FIG. 1 shows the use of two wicks, but the present invention may be practiced using a different number of wicks as one of ordinary skill in the art will understand.

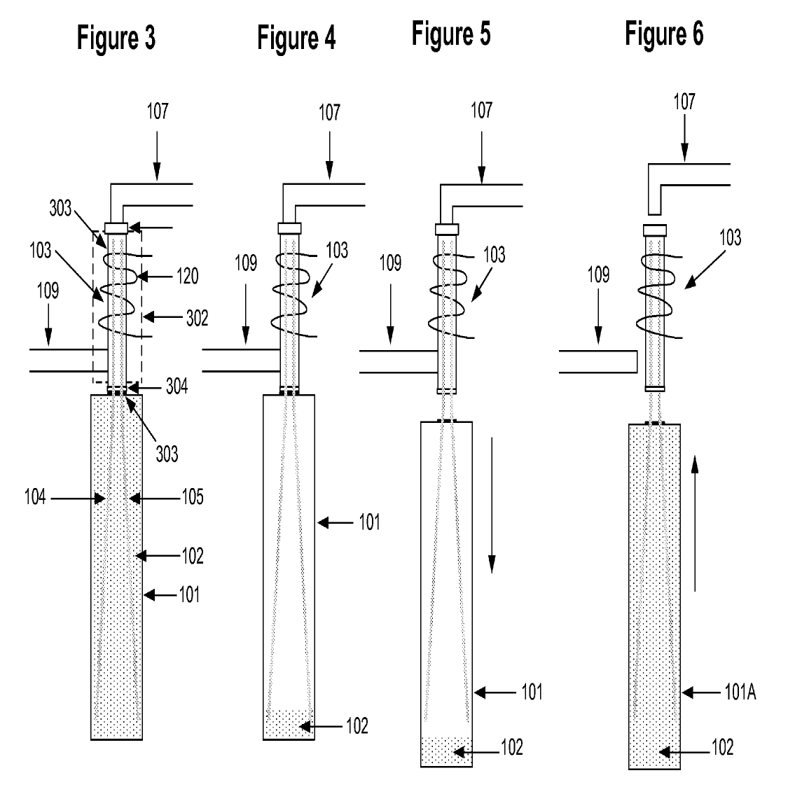

As liquid 102 is drawn into vaporizing chamber 103 by wicks 104 and 105, it is rapidly heated to its boiling point and vaporized. The internal temperature of chamber 103 is raised to the boiling point of liquid 102 by heating element 120 under the control of control unit 114 via control lines 117 and 118. Tank 101 and chamber 103 are shown in more detail in FIGS. 3-6.

Vaporizer 100 further comprises air pump 106 which draws ambient air in from the outside environment through air inlet ports 119. Pump 106 includes air outlet port 108 to which one end of air hose 107 is attached. The other end of air hose 107 is attached to air inlet port 109 of vaporizing chamber 103. When air pump 106 is energized, air flows through chamber 103 to outlet port 112 via exhaust hose 110 as indicated by arrows 121 and 122. The operation of air pump 106 also is controlled by control unit 114 via control lines 115 and 116.

As further shown in FIG. 1, vaporizer 100 is contained within housing 134A/134B. The housing is of hinged construction with a main body 134A and a cover 134B that are held together by hinges 130 and 131. For use out of doors, housing 134A/134B may be made of weather-proof construction in order to protect the interior components of the vaporizer from the adverse effects of outdoor weather. Cover 134B includes latch 135 for securing the cover in a closed position on main body 134A.

Control unit 114 includes displays 133 for displaying status information regarding the operation of vaporizer 100. Control unit 114 also controls, and is responsive to, the operation of selection buttons 132. These buttons may be used to select various operating modes and conditions of the vaporizer.

Control unit 114 further comprises On/Off status indicator 125 which indicates whether vaporizer 100 is in an “on” or “off” state and low battery status indicator 126 which indicates the state of charge of battery 136. Battery 136 is used to provide electrical power to the various components of the vaporizer, such as to control unit 114, air pump 106 and heating element 120, via power leads 128 and 129.

On/Off switch 127 is provided for turning the vaporizer on and off.

In the continuous mode of operation of vaporizer 100, control unit 114 commands heating element 120 to increase and maintain the internal temperature of vaporizing chamber 103 to the boiling point of liquid 102. The boiling point temperature vaporizes the liquid that is present in chamber 103 at the ends of wicks 104 and 105. As the liquid is vaporized in the chamber, it is replaced by the wicking action of wicks 104 and 105, which draws from tank 101 a constant supply of liquid.

Control unit 114 also commands air pump 106 to supply a constant flow of air through chamber 103, which causes the continuously vaporized liquid 102 to be blown out of chamber 103. As shown in FIG. 1, chamber 103 includes an outlet port 111 to which one end of exhaust hose 110 is attached. The other end of hose 110 is connected to exhaust port 112.

The continuous mode of operation of vaporizer 100 allows constant delivery of a scented vapor to the outside air. The particular scent will depend on the nature of liquid 102. Many man-made and naturally occurring liquids are known in the art which may be used by the electronic vaporizer of the present invention.

In the cycle mode of operation of the present invention, control unit 114 commands heating element 120 and air pump 106 to turn on for controlled periods of time. In this mode, heating element 120 is commanded to rapidly increase the temperature of vaporizing chamber 103 to the boiling point of liquid 102, followed by a command to air pump 106 to inject a burst of air of controlled duration into chamber 103. The burst of air forces the now vaporized liquid out of chamber 103 to outlet port 112 as previously described. Heating element 120 and air pump 106 are then commanded to turn off and the vaporizer remains in a quiescent state until the next cycle.

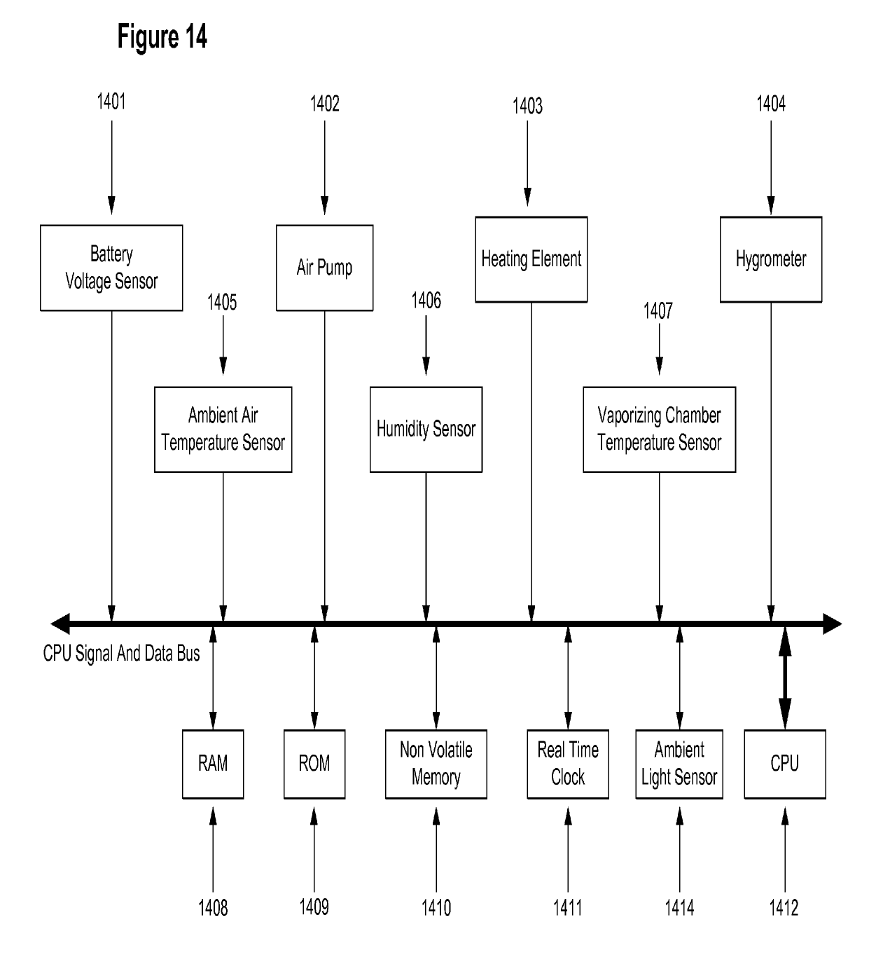

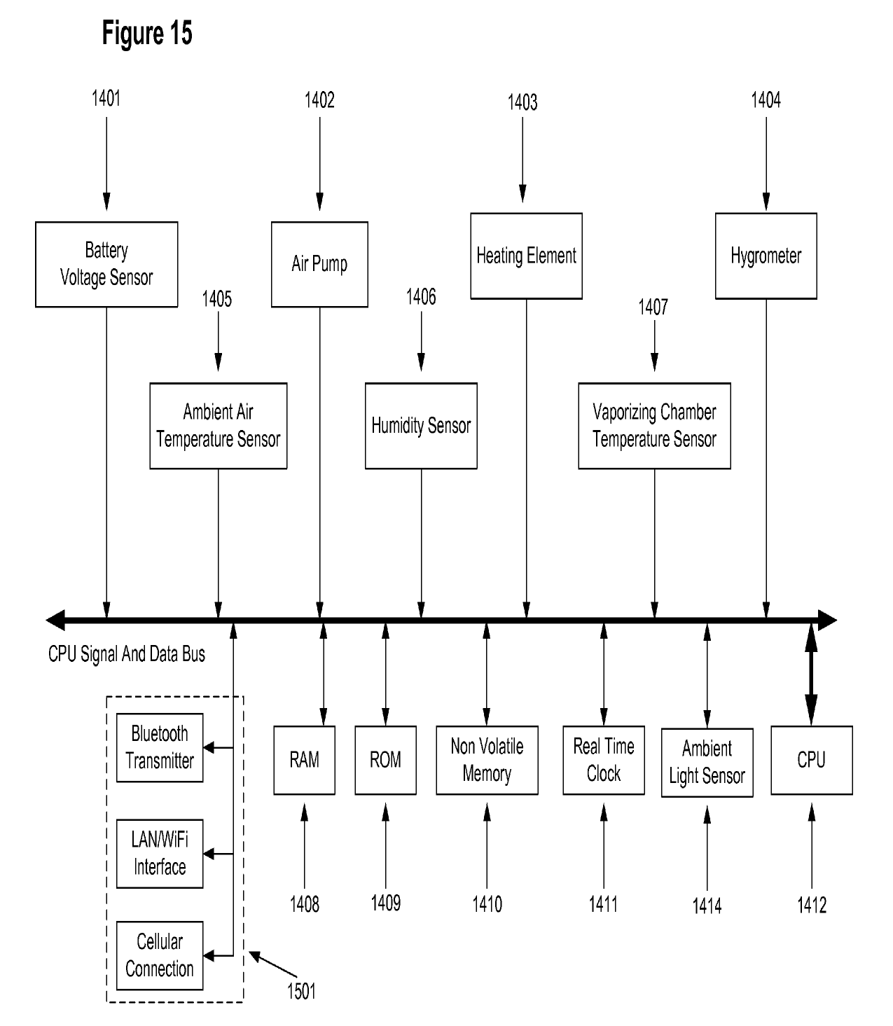

The frequency and duration of cycles can be repeated as necessary and their duration and time interval between cycles can be determined in advance and programmed into control unit 114. Moreover, as shall be described with reference to FIGS. 14 and 15, control unit 114 can dynamically determine the ideal cycle duration and time interval based on present environmental conditions. The vaporizer can evaluate the surrounding environmental conditions and automatically determine the most effective cycle frequency and duration for current conditions.

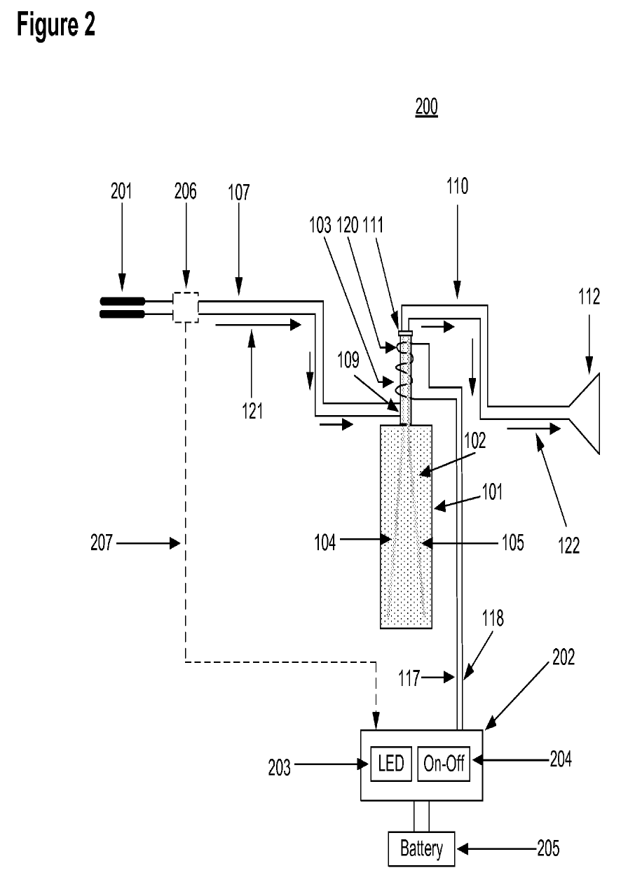

FIG. 2 illustrates another embodiment of a vaporizer 200 in accordance with the present invention. This embodiment allows a manual mode of operation of the vaporizer. In FIG. 2, the same reference numbers are used for corresponding elements in FIG. 1.

In the manual mode of operation, control unit 202 comprises an on-off switch 204, which when in the “on” position, connects battery 205 to heating element 120 via control lines 117 and 118. Element 120 increases the internal temperature of vaporizing chamber 103 to the boiling point of liquid 102 for as long as switch 204 is in the “on” state. The “on” state of heating element 120 is indicated by LED 203.

LED 203 may be a multi-color LED, wherein, for example, the color “Green” indicates that battery 205 is sufficiently charged to allow heating element 120 to increase the temperature of chamber 103 to the required vaporization temperature of liquid 102. The color “Red” may indicate that battery 205 is not sufficiently charged. Other color combinations and methods of indicating the status of battery 205 will become apparent to those of ordinary skill in the art without departing from the true scope of the present invention.

In the manual mode of operation, the user blows into mouthpiece 201 to cause a flow of air through vaporizing chamber 103 and out through outlet port 112. The manual mode of operation avoids the need for pump 106 as shown in FIG. 1.

An airflow switch, or sensor, 206 may also be used which allows heating element 120 to turn on only when a movement of air is detected from mouthpiece 201 via control line 207. The use of airflow switch 206 minimizes energy consumption of battery 205, thus allowing longer use of the vaporizer between battery charges.

FIGS. 3-6 are enlarged views of vaporizing chamber 103 and tank 101, with reference numbers referring to the same corresponding elements in FIG. 1.

As shown in FIG. 3, chamber 103 may also include a housing 303, which is formed of a material that can withstand the vaporizing temperature of liquid 102 without structural deterioration as one of ordinary skill in the art can determine. The vaporizing point typically is the boiling temperature of a liquid. As previously described, liquid 102 is drawn into vaporizing chamber 103 by wicks 104 and 105. Wicks 104 and 105 extend from the interior of tank 101 into chamber 103.

Electric heating element 120 is formed around housing 303 and when energized, heats the small quantity of liquid 102 contained in the wick ends present in chamber 103 to its vaporizing point. Heating element 120 is formed of a wound wire or coil and may be made of nickel-chromium alloy wire, platinum wire or other materials having similar properties.

A thermal cover 302 may be formed around heating element 120 as thermal insulation for chamber 103 as one of ordinary skill in the art would know how to achieve.

Vaporizing chamber 103 includes an attachment port 303 for attaching tank 101. The attachment port may be of the threaded type, compression or other type as one of ordinary skill in the art will know.

Also provided is a one-way check valve 304 which restricts air from air pump 106, or from a user blowing into mouthpiece 201 as illustrated in FIG. 2, from entering tank 101. Thus, check valve 304 allows the full force of the airflow to be directed into chamber 103 in order to effectively force vaporized liquid 102 out of chamber 103 and into exhaust hose 107 and out port 112 as shown in FIG. 1. Check valve 304 may be incorporated as part of vaporizing chamber 103 or as a part of tank 101.

As the vaporizer of the present invention is used, liquid 102 is consumed as indicated in FIG. 4. When the liquid requires replenishment, tank 101 may be disconnected from vaporizing chamber 103, as shown in FIG. 5, and a fresh tank 101A connected to chamber 103, as shown in FIG. 6.

In accordance with the present invention, tank 103 may also be refilled with liquid 102 and the same tank reconnected to vaporizer 103.

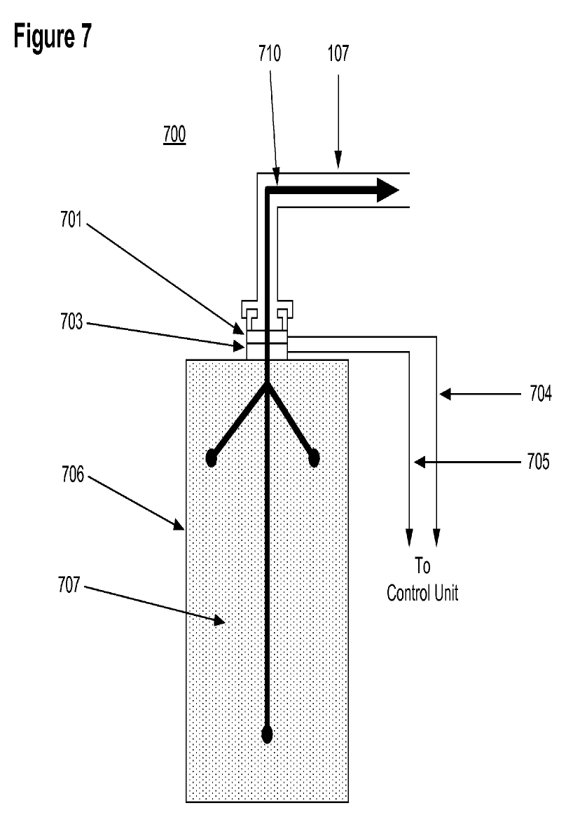

In the embodiment of the invention shown in FIG. 1, air pump 106 draws in ambient air from the outside of the vaporizer unit through air inlets 119. An alternative embodiment of air pump 106 is illustrated in FIG. 7.

As shown in FIG. 7, this embodiment of an air pump 700 is comprised of a canister 706 of compressed gas 707. Gas 707 is maintained under pressure in canister 706 by an electronically controlled one-way check valve 701. The opening and closing of the check valve is under the control of control unit 114 shown in FIG. 1 via control lines 704 and 705.

Check valve 701 can be commanded to open by control unit 114 to allow compressed gas 707 to be released into air hose 107 for entry into vaporizing chamber 103, as illustrated by arrows 121 and 122 as described with respect to FIG. 1. When all of gas 707 has been consumed, or when its pressure within canister 706 reaches a level below that required to produce an air flow of sufficient size and strength through vaporizer 103, canister 706 may be replaced with a fresh canister of compressed gas 707.

Moreover, canister 706 may also be recharged by injecting, for example, ambient air under pressure into canister 706, similar to pumping up a football or basketball, through one-valve 701.

As compressed gas 706 is released during operation of the vaporizer, its pressure within canister 706 will gradually decrease as mentioned above. Accordingly, the length of time that one-way valve 701 must remain open in order to achieved the required air flow through vaporizer 103 will depend on the air pressure within canister 706. Thus, pressure gauge 703 is provided to sense air pressure and to provide a pressure signal to control unit 114 via sense lines 704 and 705. Using the pressure signal, control unit 114 can accurately determine the proper length of time for check valve 701 to remain open based on the current air pressure within canister 706.

The alternative embodiment of air pump 700 shown in FIG. 7 also offers the ability to select the type of gas to be injected into vaporizing chamber 103 for the most effective discharge of vaporized liquid 102 into the outside air.

Injecting cold ambient air from the outside environment into vaporizing chamber 103 may lead to an undesirable cooling of the chamber. Such cooling might lessen the effectiveness of the vaporizing process.

The cooling effect can be eliminated or greatly mitigated by raising the temperature of heating element 120 to a higher level. Doing so, however, requires more electrical current from battery 136 shown in FIG. 1, and thus reduces battery life. Use of a container of compressed gas, which is relatively immune from the effects of ambient air temperature, avoids the cooling problem.

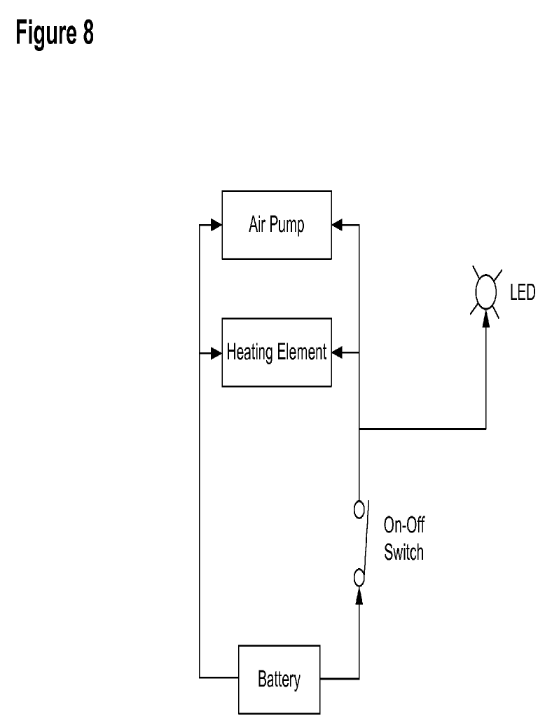

FIG. 8 is a block diagram of one embodiment of control unit 114 as shown in FIG. 1. This embodiment of control unit 114 implements a continuous mode of operation of the vaporizer, wherein the vaporizing heating element and air pump remain on constantly while the power switch is on.

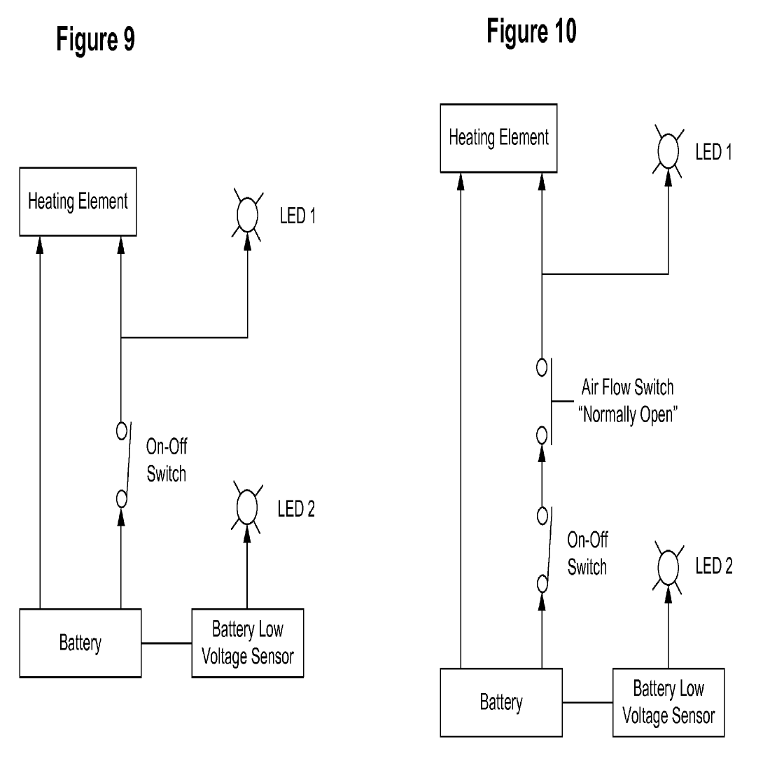

FIG. 9 is a block diagram of a one embodiment of control unit 204 as previously described with respect to FIG. 2. The block diagram in FIG. 10 includes the addition of air flow switch 206 as also shown in FIG. 2.

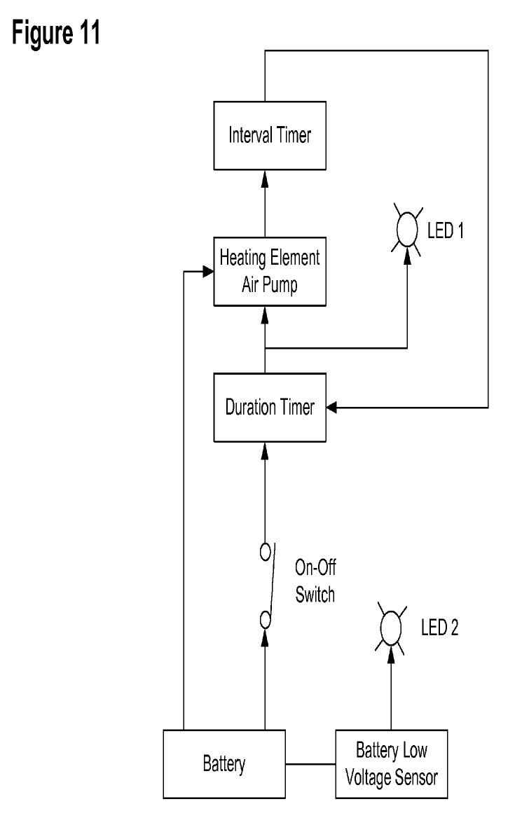

FIG. 11 is a block diagram of a further embodiment of control unit 114 shown in FIG. 1 which implements a cycle mode of operation of the present invention.

When the On-Off switch is turned on in this embodiment, heating element 120 and air pump 106 shown in FIG. 1 (or air pump 700 shown in FIG. 7—by opening check valve 701) are turned on for a first predetermined duration set by the Duration Timer. LED 1 also illuminates indicating that the heating element and air pump are energized. At the conclusion of the first predetermined duration, the heating element, air pump and LED 1 are turned off and remain off for a second predetermined duration set by the Interval Timer. At the conclusion of the second predetermined duration, the cycle repeats until the vaporizer is turned off.

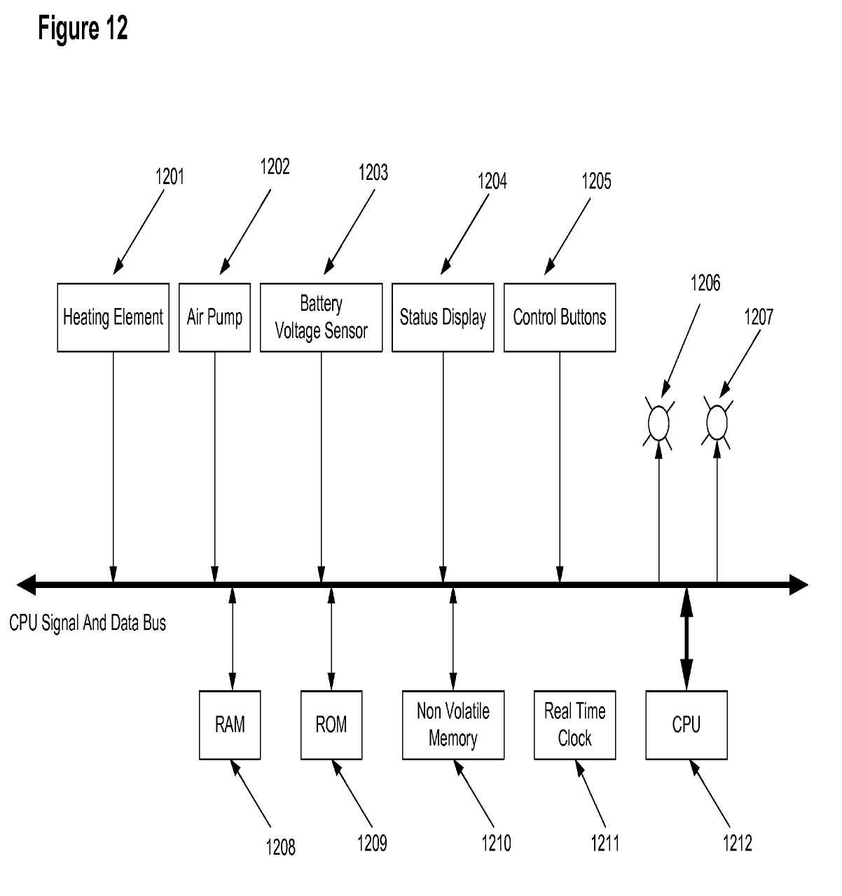

FIG. 12 is a block diagram of a further embodiment of a control unit 114 as shown in FIG. 1. The control unit includes CPU 1212 which is used for executing computer software instructions as is known in the art.

A number of elements are connected to, and controlled by, CPU 1212 via CPU Signal And Data Bus, such as heating element 1201, air pump 1202, battery voltage sensor 1203, status display 1204 and control buttons 1205.

CPU 1212 also controls the operation of low battery indicator 1206 and on/off status indicator 1207.

CPU 1212 is coupled to a number of other elements via the CPU BUS that are required for its operation. These elements include RAM 1208 (Random Access Memory) which may be used to store computer software instructions, ROM 1209 (Read Only Memory) which may also be used to store computer software instructions, and Non Volatile Memory 1210 which may be used to store computer software instructions as well.

In one aspect of the present invention, the computer software instructions that are executed by CPU 1212 are divided into two or more separate and distinct categories. These categories are stored in RAM 1208, ROM 1209 and/or Non Volatile Memory 1210. For example, a basic set of low level operating instructions, known in the art as firmware, might be stored in ROM 1209. These low level rudimentary instructions provide the necessary instructions for how CPU 1212 communicates with the various elements of vaporizer 100. Such instructions are necessary for CPU 1212 to perform any useful operations, regardless of the task being performed.

A higher level instructions set, often known in the art as “application software” operationally “sits” on top of the firmware instruction set and is used to perform specific tasks, such as receiving sensor information from battery voltage sensor 1203, reading and responding to the state of control button 1205 and controlling the operation of status display 1204, on/off status display 1206 and low battery voltage display 1207. The application software resides in Non Volatile Memory 1210.

As further shown in FIG. 12, control unit 114 includes a real time clock 1211 for keeping track of the time of events and the elapsed time between events.

In executing the firmware and application software instructions, CPU 1212 will often need to temporarily store data and intermediate calculations. Such data and intermediate calculations are stored in RAM 1209.

As is known in the art, firmware is permanently stored in ROM and is not intended to be changed. Application software also persists in Non Volatile Memory but can be changed and updated as old features in the software are deprecated and new features are added. This allows the vaporizer to be reprogrammed as needed when new software becomes available; software updates are needed or the vaporizer must be programmed for specific situations.

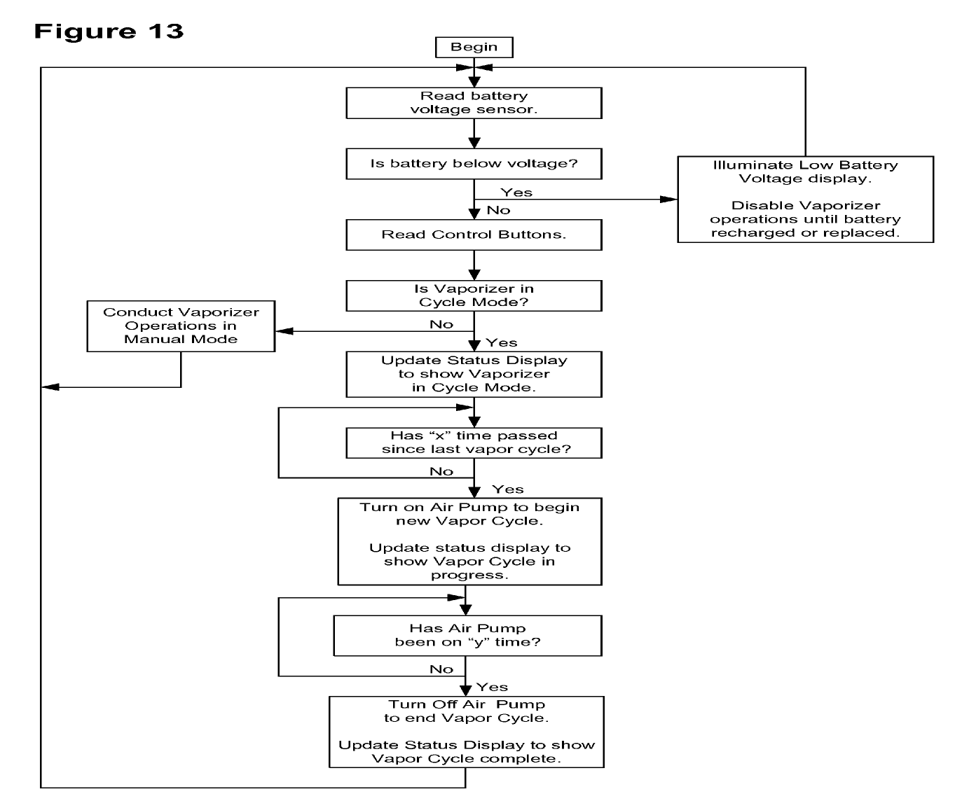

The electronic vaporizer can operate in both a manual and automatic mode with respect to frequency and duration of the vaporization process. FIG. 13 is a flow chart illustrating the operation of control unit 114.

As previously discussed, evaporation is the process of a substance in a liquid state changing to a gaseous state due to an increase in temperature and/or pressure. The rate at which a substance evaporates is related to the relative humidity of the surrounding air. Relative humidity is defined as the amount of moisture in the air compared to what the air can absorb at its current temperature. The amount of water vapor in the air at any given time usually is less than that required to saturate the air.

Evaporation rate is related to the heat index. In human terms, the heat index takes into account air temperature and relative humidity in an attempt to determine the air temperature that a human perceives. The human body cools itself by sweating and heat is removed from the body by the sweat evaporating.

High relative humidity reduces the evaporation rate because the higher vapor content of the surrounding air does not allow the maximum amount of evaporation from the body to occur. This results in a lower rate of heat removal from the body and is the reason why humans perceive a higher air temperature when humidity is high. It is known in the art that relative humidity can be measured using a hygrometer. These devices work by monitoring an electric current that is affected by moisture levels.

FIG. 14 is a further embodiment of a control unit 114 in accordance with the present invention. In addition to battery voltage sensor 1401, air pump 1402, heating element 1403, RAM 1408, Rom 1409, Non Volatile Memory 1410, Real Time Clock 1411, this embodiment of the invention includes a number of sensors that allow the electronic vaporizer of the present invention to operate in a more effective manner by taking into account the current condition of the air.

For example, vaporizing chamber temperature sensor 1407 monitors the temperature within the chamber and allows CPU 1412 to always heat the liquid to the ideal vaporization temperature. For maximum effectiveness, vaporizing temperature and duration of the vaporizing cycle, for example, must be controlled in relation to the state of the environment around which the vaporizer will be used.

Thus, a number of environment sensors in the form of ambient air temperature sensor 1405, humidity sensor 1406 and hygrometer 1404 are provided. These sensors allow CPU 1412 to calculate the relative humidity of the air and thus evaporation rate. These determinations are then used to precisely control vaporization temperature of the liquid and the duration of the vaporization period and its frequency.

For example, if the relative humidity is high, evaporation rate will be low. Thus, more frequent vaporization periods of longer duration might be necessary. Correspondingly, if relative humidity is low, fewer vaporization periods of shorter durations will still be effective. There will also be those occasions when the evaporation rate is so low that vaporization periods of any frequency and duration will not be effective. Thus, the user may, in those conditions, not wish to use the vaporizer and avoid wasting the scented liquid.

Ambient light sensor 1414 also is provided. Light sensor 1414 and real time clock 1411 may be used to measure the length of daylight during a twenty-hour period. Over a period of time and multiple measurements, the data obtained can be used to determine the previously mentioned photoperiod. Thus, this data may also be used as an additional data point for control unit 114 in determining the most effective operation of the vaporizer.

FIG. 15 is a further embodiment of the control unit of the present invention.

In this embodiment, a communications gateway 1501 is provided. The gateway allows the vaporizer to be remotely controlled through a Bluetooth connection, a portable LAN/WiFi or cellular connection. Thus, a smart device, such as a smart phone or tablet computer running an appropriate software app, can be used to control and report the status of all aspects of the vaporizer through the control unit.

While the foregoing specification teaches the principles of the present invention, with examples provided for the purpose of illustration, it will be appreciated by one skilled in the art from reading this disclosure that various changes in form and detail can be made without departing from the true scope of the invention.

I claim:

1. An apparatus for extending the range and effectiveness of scents, said apparatus comprising:
a container containing a scent;
a controllable heat source for heating said scent to a vaporized state;
a controllable air supply for blowing said vaporized scent into the environment; and
a control unit for controlling the operation of said heat source and said air supply.
